(19) United States
(12) Patent Application Publication
(10) Pub. No.: US 2013/0006335 A1
(43) Pub. Date: Jan. 3, 2013

(54) Thermal Therapy System

(75) Inventor: Mark H. Lowe, Danville, CA (US)

(73) Assignee: CoolSystems, Inc., Concord, CA (US)

(21) Appl. No.: 13/441,770

(22) Filed: Apr. 6, 2012

Related U.S. Application Data
(60) Provisional application No. 61/472,596, filed on Apr. 6, 2011.
(60) Provisional application No. 61/472,598, filed on Apr. 6, 2011.
(60) Provisional application No. 61/472,602, filed on Apr. 6, 2011.

(51) Int. Cl. A61F 7/00; A61F 7/08

(52) U.S. Cl. 607/104; 607/112; 607/108; 607/109; 607/111

(57) ABSTRACT

A thermal therapy system for providing thermal treatment to a body requiring treatment. The system may include a plurality of therapy wraps, each configured to exchange heat with the body. The therapy wraps may be secured to different locations on a substrate and unfurled onto a rigid board for supporting the body. A control unit may be configured to independently control the therapy wraps. The therapy wraps may have a plurality of layers. One of the layers is a heat exchange layer comprising a heat transfer device for exchanging heat with the body. One of the layers may be a compressive layer for applying a compressive force to the body. One of the layers may be a structural layer including a rigid structural member. Also disclosed are methods of administering a temperature-controlled treatment to an animate body.

### CROSS REFERENCE TO RELATED APPLICATIONS

This application claims priority to U.S. Provisional Application No. 61/472,596, filed on Apr. 6, 2011; U.S. Provisional Application No. 61/472,598, filed on Apr. 6, 2011; and U.S. Provisional Application No. 61/472,602, filed on Apr. 6, 2011, which are herein incorporated by reference in their entireties.

### FIELD OF THE INVENTION

The present invention relates generally to therapy of an animate body, and more particularly a system for providing thermal therapy to a mammal.

### INCORPORATION BY REFERENCE

All publications and patent applications mentioned in this specification are herein incorporated by reference for all purposes to the same extent as if each individual publication or patent application was specifically and individually indicated to be incorporated by reference.

### BACKGROUND OF THE INVENTION

Temperature-controlled therapy has long been practiced for physical therapy, sports injuries, and other settings. Thermal therapy commonly includes cooling, heating, and/or applying compression to a traumatized area of a human body to facilitate healing and prevent unwanted consequences of the trauma. This form of therapy is commonly referred to as RICE (Rest, Ice, Compression and Elevation). RICE is also commonly used in sports medicine to reduce the risk of long-term damage to muscles and joints and/or alleviate pain and soreness.

Conventional temperature-controlled therapy involves applying ice bags or the like to a treatment area to provide deep cooling. Elastic wraps are often applied over the bags to keep them in place and provide compression to the body part. Ice bags and elastic wraps lack control and usually require a user to put the bag on and off to adjust cooling.

More sophisticated animate body heat exchangers have been developed recently. Thermal therapy systems commonly include a heat exchanger, a control unit for the heat exchanger, and a sleeve for positioning the heat exchanger on a body part to be treated. The control unit regulates delivery of a heat exchange fluid to the heat exchanger for circulation through a fluid bladder. Many systems also include a compressive mechanism such as a compliant gas pressure bladder that overlays the fluid bladder. The gas pressure bladder directs a compressive force to the fluid bladder to press the bladder against the body part to be subjected to heat exchange and apply compression to the body part to reduce edema.

There has been a focus with existing therapy wrap designs on improving conformance to body parts. Better conformance generally leads to improved therapy performance and the ability to use therapy wraps in a greater array of applications.

There is a need for thermal therapy wraps with better conformance properties. There is the need for thermal therapy wraps applicable to more treatment settings and patient populations. There is a continuing need for therapy wraps with improved heat exchange performance. There is the need for therapy wraps that can be applied to a wide variety of anatomical shapes. There is a need for a therapy wrap that achieves better apposition to the body. There is a need for a temperature-controlled therapy system with improved patient comfort and/or reduced risks of injury to the body part treated. There is a need for improved systems and methods for heating, cooling, and/or compressing a body in need of treatment.

These and other problems are overcome by the invention disclosed herein.

### SUMMARY OF THE INVENTION

The present invention involves improvements in heat transfer therapy apparatus and avoids disadvantages in the prior art.

Various aspects of the invention are directed to a system for providing thermal treatment to a body requiring treatment, the system comprising at least one therapy wrap comprising a heat exchange layer configured to exchange heat with a body; and a rigid substrate for supporting the therapy wrap.

In various embodiments, the at least one therapy wrap is secured to the rigid substrate. In various embodiments, the at least one therapy wrap is secured to a flexible substrate configured to be unfurled onto the rigid substrate. In various embodiments, the rigid substrate is a backer board or a brace.

In various embodiments, the heat exchange layer comprises at least one a fluid bladder configured to circulate a heat transfer fluid. In various embodiments, the at least one therapy wrap further comprises an insulating layer positioned between the heat exchanger layer and the body to receive treatment; and a compressive layer for applying a compressive force to the body, the compressive layer positioned on an opposite side of the heat exchange layer as the insulating layer. In various embodiments, the therapy wrap further comprises a compressive layer including an expandable gas bladder portion for applying a compressive force to the body, the heat transfer device, or both, the compressive layer positioned outwardly of the heat exchange layer. In various embodiments, the expandable portion includes a gas bladder and/or expandable foam.

In various embodiments, the system includes a plurality of therapy wraps each positioned at different locations on the substrate corresponding to different parts of the body. The plurality of therapy wraps may be configured to exchange heat with the animate body at different rates.

In various embodiments, each of the plurality of therapy wraps comprises a heat exchange layer including at least one a fluid bladder configured to circulate a heat transfer fluid, the system further comprising a control unit configured to independently regulate the flow of heat transfer fluid in each of the plurality of therapy wraps.

Various aspects of the invention are directed to a therapy wrap for providing thermal treatment to a body requiring treatment, the therapy wrap comprising a first layer for contacting the body at a treatment site; and a heat exchange layer positioned outwardly of the first layer, the heat exchange layer including at least one heat transfer device configured to circulate a heat transfer fluid to exchange heat with the body. The first layer may include a cut-out in a location corresponding to a feature of the body. In various embodiments, the first layer comprises padding. In various embodiments, the therapy wrap further comprises a structural layer including a rigid member for increasing local bending strength of the therapy wrap. In various embodiments, the rigid member extends along a region of the cut-out, wherein the rigid member has a predetermined shape corresponding to the feature. In various embodiments, one of the plurality of therapy wraps is configured to administer therapy at a temperature significantly below a normal internal temperature of the body and another of the therapy wraps is configured to administer therapy at a temperature warmer than the normal internal temperature.

The wrap and method of the present invention have other features and advantages which will be apparent from or are set forth in more detail in the accompanying drawings, which are incorporated in and form a part of this specification, and the following Detailed Description of the Invention, which together serve to explain the principles of the present invention.

### BRIEF DESCRIPTION OF THE DRAWINGS

FIG. 1 is a schematic diagram of a thermal therapy system in accordance with the invention, the system comprising a backer board and a plurality of therapy wraps.

FIG. 2A is a cross-sectional view of a portion of a therapy wrap for use with the system of FIG. 1, illustrating multiple layers of the therapy wrap.

FIG. 2B is a cross-sectional view of a portion of another therapy wrap for use with the system of FIG. 1, illustrating multiple layers of the therapy wrap.

FIG. 2C is a cross-sectional view of a portion of another therapy wrap of the system of FIG. 1, illustrating a cut-out section corresponding to an anatomical body part.

FIG. 3 is an enlarged portion of a corner of a therapy wrap for use with the system of FIG. 1, illustrating an edge of the top layer of the wrap peeled back.

FIG. 4 is a top plan view of a therapy wrap similar to the wrap of FIG. 2C, illustrating a centrally-located cut-out section and a fluid pathway directed around the cut-out section.

FIG. 5 is a cross-sectional view of a therapy wrap for use with the system of FIG. 1, illustrating the therapy wrap in a rolled configuration for transport.

FIG. 6 is a perspective view of several therapy wraps in accordance with the invention, the therapy wraps comprising a heat exchanger and a loose overlayer.

FIG. 7 is a simplified top plan view of a therapy wrap in accordance with the invention, the wrap comprising different closure members.

### DETAILED DESCRIPTION OF THE INVENTION

Before the present invention is described, it is to be understood that this invention is not intended to be limited to particular embodiments or examples described. Further, when referring to the drawings, like numerals indicate like elements.

Unless expressly stated otherwise, the terms used herein are to be understood as used by one of ordinary skill in the art. In various respects, use of the singular in connection with the terms herein includes the plural and vice versa.

“Body” is to be understood as used in the medical and biological fields and generally includes any animate body including, but not limited to, mammals. In various respects, “body” refers to human or equine patients. In various respects “body part” and “body” are used interchangeably. In various respects, “body part” refers to a part of a body in direct communication with a therapy system as described herein.

“Core cooling” is to be understood as generally used in the medical and biological fields, and in various respects, refers to application of cooling therapy to decrease the body temperature. In various respects, “core cooling” refers to cooling of the internal body temperature of a human below 95 degrees Fahrenheit, and in various respects below 90 degrees Fahrenheit.

“Body temperature” and “internal body temperature” refer to the internal temperature or core temperature of a respective body as understood in the medical art.

As used herein, the “average temperature” of the wrap refers to the average of the wrap inlet temperature and the wrap outlet temperature.

“Temperature delta” refers to the difference between the wrap outlet temperature and the wrap inlet temperature. One will appreciate that in some cases the temperature delta through the wrap depends on the fluid flow rate, the heat load, and the specific heat of the thermal fluid.

“Maximum temperature” and “minimum temperature” generally refer to the maximum and minimum temperatures in a respective element, and in various respects, within the fluid bladder of the wrap.

“Heat transfer fluid” is to be understood as generally used in the art, and in various respects, refers to the fluid circulated in the heat transfer device for exchanging heat with the subject animate body. “Heat transfer fluid”, “heat transfer medium”, “heat exchange medium” and “heat exchange fluid” are used somewhat interchangeably. In various respects, “heat exchange medium” refers to a medium or cooling source through which the heat transfer fluid is passed to lower its temperature before circulating in the heat transfer device.

In various respects, “inner” and “inwardly” refer to a direction towards a target body part and “outer” and “outwardly” refer to a direction away from the body part.

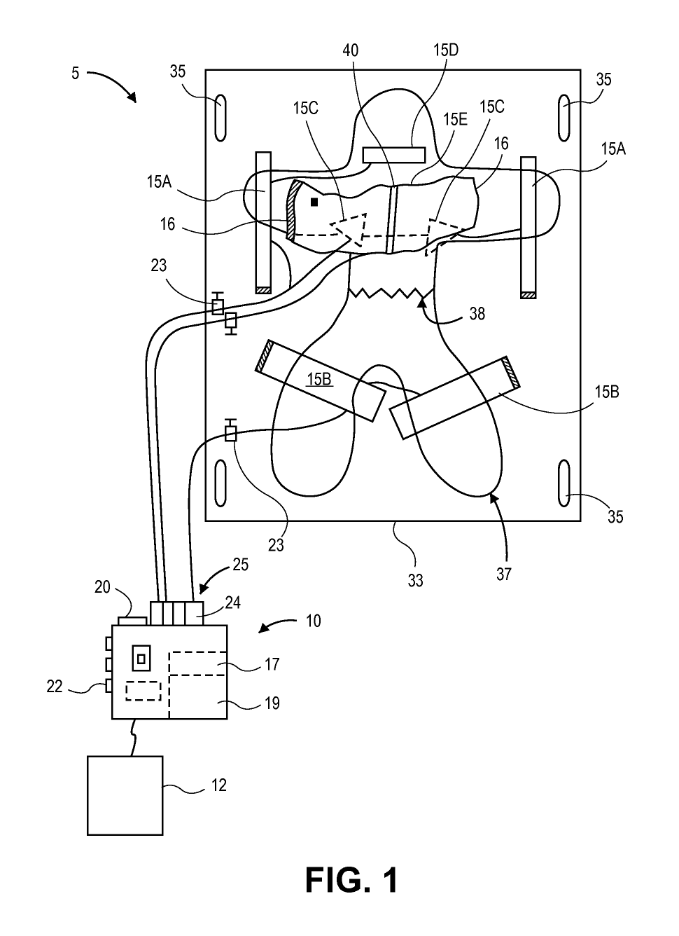

FIG. 1 illustrates a representative number and type of components used in an exemplary thermal therapy system, generally designated 5, in accordance with various aspects of the invention. The thermal therapy system is configured for administering temperature-controlled therapy to a body including, but not limited to, the application of cooling, heating, and/or compression. Exemplary system 5 includes a control unit 10, a power source 12, and a plurality of thermal therapy wraps 15.

Control unit 10 is similar in many respects to existing control units. The exemplary control unit includes a pressurized gas source 17, a heat transfer fluid source 19, a control panel 20, a communications port 22, and an input panel 25 having fluid and gas ports 24. The exemplary heat transfer fluid source is a reservoir for supplying cooled heat transfer fluid, such as water. The heat transfer fluid source may be configured to supply heated fluid. The exemplary pressurized gas source is a gas compressor for supplying a compressed gas, such as air. The control panel may include a user interface, such as an output display, keyboard, a knob, a button, and the like. The communications port may include a wireless communications device, a computer network port, a serial port, a disk drive, and the like. One will appreciate that various modifications may be made to the control unit configuration in accordance with the invention.

The therapy wraps are connected to the control unit using conventional structures such as hoses and valves. The wraps may be connected in serial (e.g. a daisy chain), in parallel, or a combination of the same. The exemplary system includes a pump in fluid communication with coolant source 19, control unit 10, and therapy wrap 15. The pump delivers heat transfer fluid from the coolant source to the therapy wrap through ports in a manifold connector. In general, the pump is controlled by the control unit using a control signal. The manifold connector is similar in many respects to the three-port manifold connector disclosed by U.S. Pat. No. 6,871,878, the entire contents of which is incorporated herein for all purposes by reference. Other suitable manifold constructions are disclosed in U.S. Pat. Nos. 5,104,158 and 5,052,725, both to Meyer, et al. and both hereby incorporated herein for all purposes by reference. The system may also include valving for controlling the fluid flow. The exemplary system includes shut-off valves 23 for opening and closing the fluid and/or gas lines to the therapy wraps.

Control unit 10 includes an input panel 25 corresponding to the fluid, gas, and/or electrical lines from the therapy wrap. The control unit input panel may be configured as an easy quick connect similar to the manifold connector of the therapy wraps. Each of the connectors may have fluid ports, a gas port, and an electrical connection. It should be understood that other manifold configurations and/or couplings to define a flowpath between the coolant source and the bladders can be used as would be apparent to one of skill in the art.

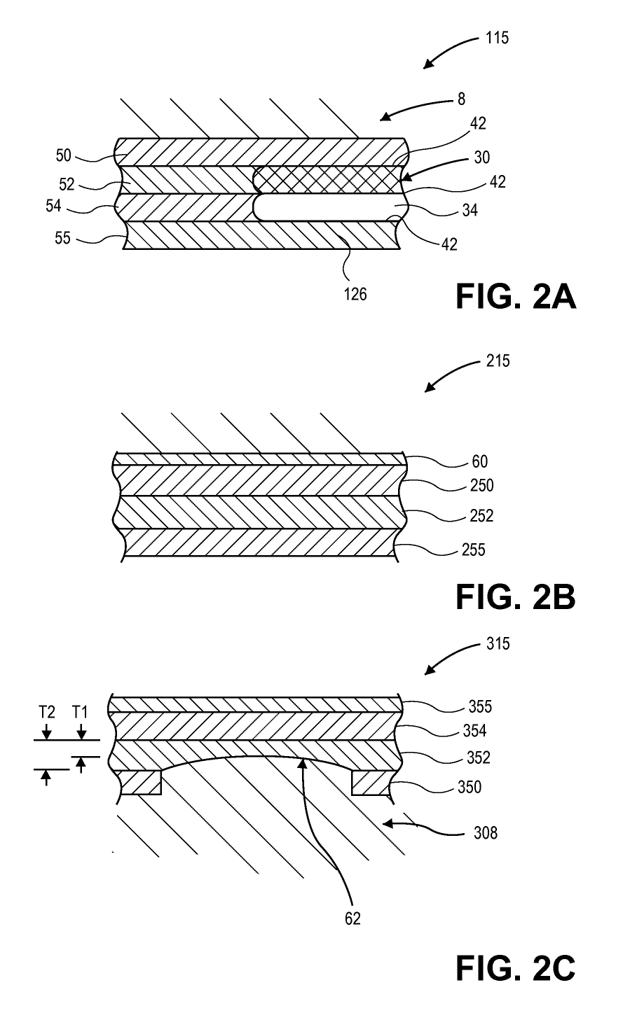

With reference to FIGS. 1 and 2A, the control unit controls the function of therapy wraps 15. The therapy wrap may include a sleeve cover for receiving a heat exchange device 30. The heat exchange device may include a heat exchanger and a compressive mechanism. In an exemplary embodiment, the heat exchanger is a fluid bladder 32 for circulating the heat transfer fluid from source 19. The exemplary compressive mechanism is a gas pressure bladder 34 for receiving gas from source 17.

Therapy wrap 15 is configured for wrapping to a portion of an animate body for delivering treatment. Various aspects of the therapy wrap may be similar to the devices disclosed by U.S. Pat. No. 7,107,629 to Miros et al. and U.S. Patent Pub. No. 2005/0256556 Al to Schirrmacher et al., the entire contents of which are incorporated herein for all purposes by reference. The fluid bladder is adapted exchange heat with an adjacent body when the fluid is circulated in the bladder. The body may include, but is not limited to, a mammalian body such as a human or an equine animal. The exemplary therapy wrap is in the form of a sleeve for connecting various components of heat exchange device 30 to the patient's body.

Various aspects of the invention are similar to the subject matter described in: U.S. patent application Ser. No. 09/127,256 (filed Jul. 31, 1998) entitled, “Compliant Heat Exchange Panel,” issued on Apr. 3, 2007 as U.S. Pat. No. 7,198,093; U.S. patent application Ser. No. 09/798,261 (filed Mar. 1, 2001) entitled, “Shoulder Conformal Therapy Component of an Animate Body Heat Exchanger,” published on Aug. 30, 2001 as U.S. Publication No. 2001-0018604A1; U.S. patent application Ser. No. 09/901,963 (filed Jul. 10, 2001) entitled, “Compliant Heat Exchange Splint and Control Unit,” published on Nov. 8, 2001 as U.S. Publication No. 2001-0039439A1; U.S. patent application Ser. No. 09/771,123 (filed Jan. 26, 2001) entitled, “Wrist/Hand Conformal Therapy Component of an Animate Body Heat Exchanger,” published on Oct. 25, 2001 as U.S. Publication No. 2001-0034546A1; U.S. patent application Ser. No. 09/771,124 (filed Jan. 26, 2001) entitled, “Foot/Ankle Conformal Therapy Component of an Animate Body Heat Exchanger,” published on Feb. 14, 2002 as U.S. Publication No. 2002-0019657A1; U.S. patent application Ser. No. 09/771,125 (filed Jan. 26, 2001) entitled, “Conformal Therapy Component of an Animate Body Heat Exchanger having Adjustable Length Tongue,” published on Oct. 25, 2001 as U.S. Publication No. 2001-0034545A1; U.S. patent application Ser. No. 10/784,489 (filed Feb. 23, 2004) entitled, “Therapy Component of an Animate Body Heat Exchanger,” published on Aug. 26, 2004 as U.S. Publication No. 2004-0167594A1 which is a continuation of U.S. patent application Ser. No. 09/765,082 (filed Jan. 16, 2001) entitled, “Therapy Component of an Animate Body Heat Exchanger and Method of Manufacturing such a Component,” issued on Feb. 24, 2004 as U.S. Pat. No. 6,695,872 which is a continuation-in-part of U.S. patent application Ser. No. 09/493,746 (filed Jan. 28, 2000) entitled, “Cap And Vest Garment Components Of An Animate Body Heat Exchanger,” issued on Jan. 30, 2001 as U.S. Pat. No. 6,178,562; U.S. patent application Ser. No. 10/122,469 (filed Apr. 12, 2002) entitled, “Make-Break Connector For Heat Exchanger,” issued on Mar. 29, 2005 as U.S. Pat. No. 6,871,878; U.S. patent application Ser. No. 10/637,719 (filed Aug. 8, 2003) entitled, “Apparel Including a Heat Exchanger,” issued on Sep. 19, 2006 as U.S. Pat. No. 7,107,629; U.S. patent application Ser. No. 12/208,240 (filed Sep. 10, 2008) entitled, “Modular Apparatus for Therapy of an Animate Body,” published on Jan. 1, 2009 as U.S. Publication No. 2009-0005841A1 which is a divisional of U.S. patent application Ser. No. 10/848,097 (filed May 17, 2004) entitled, “Modular Apparatus for Therapy of an Animate Body,” issued on Mar. 1, 2011 as U.S. Pat. No. 7,896,910; U.S. patent application Ser. No. 11/707,419 (filed Feb. 13, 2007) entitled, “Flexible Joint Wrap,” issued on Nov. 23, 2010 as U.S. Pat. No. 7,837,638; U.S. patent application Ser. No. 11/854,352 (filed Sep. 12, 2007) entitled, “Make-Break Connector Assembly with Opposing Latches,” issued on Jun. 8, 2010 as U.S. Pate. No. 7,731,244, which is incorporated herein for all purposes by reference.

The above systems generally provide active heating, cooling, and/or compression for humans and other animal bodies. They are used, for example, in physical therapy, pre-game conditioning, minor injury care, post-operative care, and emergency medical care, among other applications. Thermal therapy systems exist in a number of different forms. In general, there is a control unit, a connector hose, a therapy wrap comprising a heat transfer device and a sleeve cover, and a power source (i.e., battery or externally-powered electric source).

Therapy wrap 15 comprising a cover and heat exchange device 30 is applied to a portion of the mammal's body requiring therapy. Control unit 10 modulates the flow of heat transfer medium in the wrap to achieve the desired therapeutic result. One such system is disclosed, for example, in U.S. Pat. No. 6,178,562, the disclosure of which is herein incorporated for all purposes by reference. The therapy wrap may take different forms such as a vest, a wrist wrap, a forearm wrap, a head cap, a wrap for a body limb, a foot wrap, a leg wrap, and more. The wrap may have a variety of shapes and sizes for applying to different anatomies and/or different parts of the body as would be appreciated by one of skill from the description herein. The parts of the body to receive treatment with the wrap include, but are not limited to, all or part of a torso, a thoracic region, a cranial region, a throat region, a limb (e.g. a thigh or arm), a heart region, a lung region, a chest region, a wrist, a foot, and a combination of the same. The wrap may be configured for positioning adjacent selected portions of the patient's vascular system, for example, the heart, the femoral artery, the carotid artery, or the superior vena cava. In various respects, the therapy wrap may include components configured for applying to a small body parts such as a wrist. Aspects of the therapy wrap may be similar to the wrap disclosed by U.S. Patent Pub. No. 2001/0034546 A1 to Elkins, the entire contents of which is incorporated herein for all purposes by reference.

Exemplary control unit 10 regulates the flow of heat transfer fluid and gas to heat exchange device 30. The control unit may control and adjust the flow rate, flow pressure (including backpressure), and/or inlet temperature of the heat transfer fluid. In various embodiments, the control unit controls the fluid supply using a pump. Similarly, the control unit may control the flow rate and/or pressure (including backpressure) of the gas supplied to the heat exchange device.

Performance of thermal therapy wrap 15 may be improved by adjusting the heat transfer fluid flow rate, adjusting the heat transfer device temperature, and/or providing additional features to the thermal therapy device. In a closed loop, return flow arrangement, by example, the velocity of the fluid and the heat transfer rate are generally proportional to the flow rate. Reducing the flow rate of the fluid of a given temperature through the fluid bladder will also reduce the amount of energy removed from (or added to) the patient. Conversely, increasing the flow rate will increase the amount of energy removed from (or added to) a patient. In a cold therapy device, with the wrap applied to a mammalian body, the temperature of the fluid leaving the wrap is warmer than the temperature of the fluid entering the wrap because the mammalian body is typically warmer than the thermal fluid. The temperature from the coolant source outlet determines the inlet temperature of fluid bladder and is generally lower than the average temperature. For example, if an average wrap temperature of 5° C. is desired, then the inlet temperature may be about 4° C. In this example, the outlet temperature may be about 6° and the temperature delta across the heat transfer device may be 2° C.

In various embodiments, the heat transfer fluid at the therapy wrap inlet is maintained at a desired temperature. Generally, the desired temperature is lower or higher than the temperature expected for the body part receiving treatment. In a typical cold therapy system, the heat transfer fluid is cooled prior to the inlet to the fluid bladder by passing the fluid through coolant source 19. One such system is disclosed, for example, in U.S. Pat. No. 6,178,562, the disclosure of which is herein incorporated for all purposes by reference. In the exemplary system, the coolant source is configured to provide a cooled heat transfer fluid. Suitable cooling sources include, but are not limited to, thermoelectric- and chemical-based cooling. In various embodiments, the cooling source comprises a fluid reservoir cooled with ice, a chemical-based cooling device (e.g. a chemical cold pack), or a thermoelectric-based cooling device. In various embodiments, the temperature in the reservoir is in a range between about 40° F. and about 50° F. In various embodiments, the cooling source comprises an ice bath and water. In various embodiments, the cooling source comprises a water reservoir cooled by a refrigeration unit. Examples of cooling and heating sources are described in the patents and publications incorporated above and co-pending U.S. App. No. 61/472,596 (attorney matter no. 11185-721.100), filed Apr. 6, 2011 and incorporated herein for all purposes by reference. Other direct and indirect cooling sources may be provided such as thermoelectric, chemical, and electromechanical devices as would be understood by one of skill in the art.

The therapy wrap in accordance with the invention may have a variety of shapes and sizes for applying to different portions of the body or different body anatomies. The therapy wrap may be shaped and configured for application to a mammal such as a human. In various embodiments, the therapy wrap is shaped for applying to and covering all or part of a torso, a thoracic region, a cranial region, a throat region, a limb, and a combination of the same. In various embodiments, the body part to receive treatment is measured and the therapy wrap(s) are selected to fit the patient based on the measurements and body part. Thus, the therapy wrap is customized to the body. The system may also include a plurality of wraps for different body parts and different sizes (e.g. small, medium, and large). Thus, a user can select a somewhat customized wrap depending on the application and patient.

With continued reference to FIG. 1, exemplary system 5 includes a plurality of therapy wraps 15 for application to different parts of a body. A first pair of wraps 15a are configured for application to a wrist or forearm of the body. More specifically, the wraps are configured to apply therapy to the blood vessels in the forearm. In various respects, the wraps are similar to those described in U.S. Publication No. 2001-0034546A1 and U.S. application Ser. No. 12/910,772, the entire contents of which are incorporated herein for all purposes by reference. A second pair of wraps 15b are configured for application to a leg or thigh, and a third pair of wraps 15c are configured for application to the armpit or shoulder region. In various respects, the wraps are similar to those described in U.S. patent application Ser. No. 12/910,772, the entire contents of which are incorporated herein for all purposes by reference. The system includes another wrap 15d for applying to a neck region. A single, large wrap 15e is configured for applying to a chest region.

Any or all of the therapy wraps may include a compressive gas pressure bladder. The number, type, and location of the therapy wraps to be positioned on the body may be based on the desired treatment as would be understood by one of skill in the art. If core cooling of the body is desired, for example, multiple wraps may be provided for each of the key cooling points to lower the body temperature. Each of the multiple wraps may include one or more heat transfer devices. The selection and configuration of the wraps may be modified to enable wrapping to the target parts of the body and positioning of selected heat transfer devices in predetermined locations. For example, a heat transfer device with higher heat exchange capabilities may be selected for a chest region than a heat transfer device for an arm.

The therapy wraps of exemplary system 5 are positioned on a rigid substrate or backer board 33. The exemplary backer board is a rigid substrate or board similar to conventional stretchers. The board is configured for moving a body and includes handles 35. The backer board also includes an outline 37 for a patient. The outline may be a marker, a molded indentation, and the like. In an exemplary embodiment, the outline is a foam pad shaped to support a body. The pad includes a joint 38 configured to allow the bottom portion to separate from the top portion thereby increasing the length for taller patients. Because the therapy wraps are positioned on the backer board in predetermined positions corresponding to selected body regions, the backer board enables faster and easier application of the wraps to the body. As will be understood from the foregoing, the backer board also allows movement of the body while the wraps are in position.

Therapy wraps 15 may be attached to backer board 33 using various conventional techniques. Therapy wrap 15e is attached to the board along a centerline 40. The wrap includes two long strap sections 16 extending from the centerline. Once the body is positioned on the backer board, the flaps are pulled over the chest and fastened. Therapy wraps 15a and 15b have similar configurations except that they fasten similar to a watch strap or belt. Exemplary wrap 15d is attached like a collar. Exemplary wraps 15c are substantially flat and secured to the backer board. The exemplary wraps 15c are substantially rigid and non-compliant to enable better attachment to the backer board. Wraps 15c are configured to be sandwiched between the back of the body (e.g. the shoulder blade region) and the backer board. Whereas the other wraps enclose a body part and can apply thermal therapy to the entire circumference, wraps 15c only apply therapy to one side of the patient. In the alternative to a pre-assembled backer board with wraps attached, any or all of the therapy wraps may be provided free and independent of the backer board.

Each of exemplary therapy wraps 15a, 15b, 15c, 15d, and 15e are configured independently for applying treatment under specific and unique parameters. Wraps 15a have a narrow width and relatively small treatment area. The wraps have an internal fluid bladder with relatively narrow fluid flowpath for increasing the flow rate through the wrap (all things remaining equal, the velocity increases as the pathway area decreases with incompressible fluids). Thus, the wraps achieve a higher heat exchange rate. Wraps 15b, by contrast, are larger and configured for a slower fluid flow rate. The control unit is also configured to circulate fluid to wraps 15b at a higher temperature because, for example, the inner thigh area is more sensitive than the shoulder. Similarly, wrap 15d may be configured for circulating fluid at higher or lower temperatures than the leg region wraps.

The exemplary wraps may also be configured for application of independent compression therapy conditions. For example, therapy wrap 15e may be configured to apply less compression than wraps 15a and 15b to avoid straining the lungs. Therapy wrap 15d may be configured not to apply any compression to avoid the risk of strangulation. In various embodiments, any or all of the therapy wraps are configured for applying heat, cooling, and/or compression therapy under different conditions. In various embodiments, any or all of the therapy wraps are configured for applying heat, cooling, and/or compression therapy independently of one another. Some of the therapy wraps may be operated at one time while others are not operating.

In various embodiments, the pressure of gas furnished by the control unit to a wrap is between about 0.25 psig and about 20 psig, preferably between about 0.25 psig and about 5 psig, and more preferably about 0.25 to about 1.5 psig. In various embodiments, the control unit maintains a compressive force of between about 0.25 psig and about 5 psig. In various embodiments, the control unit maintains a compressive force of between about 0.25 psig and about 0.5 psig. In various embodiments, the pressure of gas furnished by the control unit is user selectable in increments of 5 mm Hg from 0 mm to about 75 mm. In various embodiments, the system includes a plurality of gas pressure bladders, a first gas pressure bladder at a first pressure and a second gas pressure bladder at a second pressure different than the first.

In various embodiments, the pressure of gas furnished by the control unit is based on the patient's response and/or the treatment application. For example, if the patient is wearing the wrap during exercise, the pressure may vary based on how strenuous the exercise is. If the patient is having trouble breathing, the control unit may decrease the compressive force around the lungs. The pressure profile map may be set to adjust based on a predetermined routine. In various embodiments, the pressure profile map includes 3 minutes of slowly increasing pressure followed by 2 minutes of decreasing pressure. In various embodiments, the pressure profile map includes 30 seconds of increasing pressure followed by 15 seconds of decreasing pressure. In various embodiments, the pressure fluctuates at random or based on the type of wrap. In various embodiments, the pressure profile map includes 2 minutes of compression followed by 1 minute with no compression.

Further details regarding fluid bladders, cooling sources, and their operation and manufacture are described in U.S. Pat. Nos. 7,198,093 and 6,695,872, U.S. patent application Ser. No. 12/982,266, and co-pending U.S. App. No. 61/472,596 (attorney matter no. 11185-721.100) filed Apr. 6, 2011, the entire contents of which are incorporated herein for all purposes by reference. One will appreciate that the configuration and operation parameters of the therapy wraps and system may be modified in accordance with the invention.

FIGS. 2A, 2B, and 2C illustrate cross-sections of portions of representative therapy wraps for use in the thermal therapy system of FIG. 1. The therapy wraps of FIGS. 2A, 2B, and 2C are similar in many respects to the therapy wraps disclosed above and in the patents and publications incorporated herein. Like reference numerals have been used to describe like components.

Turning to FIG. 2A, an exemplary therapy wrap 115 includes several layers of material 42 defining a flexible fluid bladder 32 through which a liquid is circulated and a gas bladder 34 in which a pressurized gas is injected. The gas pressure bladder is adapted to inhibit edema and/or for pressing the fluid bladder against the body part to be subjected to heat exchange. Exemplary therapy wrap 115 is in the form of a conventional multi-bladder assembly within a sleeve for positioning adjacent a treatment site of a body. In various aspects, the multi-bladder assembly is manufactured and configured using known techniques. Suitable materials 42 for the bladders include, but are not limited to, thermoplastics such as polyvinyl chloride (PVC), poly(urethane) (PU), polyethylene (PE), polypropylene, copolymers, and more.

The thermal bladder assembly includes compliant fluid bladder 32 which is overlaid by gas pressure bladder 34. More specifically, outer gas pressure bladder 34 is adapted to receive a first fluid such as a gas (e.g. air) that can be regulated to provide the desired amount of inflation of the bladder or pressure therein. This inflation or pressure affects the compressive force applied to the animate body during use. Inner fluid bladder 32 is adapted to receive a fluid, such as a coolant which can be in the form of a cold liquid, to transfer heat away from the animate body part. Alternatively, the fluid supplied to the inner bladder can have a temperature higher than the animate body part to heat the body part.

In FIG. 2A, therapy wrap 115 having a multi-layered configuration is shown positioned against a body 8. The exemplary therapy wrap includes a first layer 50 forming an inner surface and positioned directly adjacent the body, a second layer 52, a third layer 54, and an outer layer 55. The second layer and third layer are positioned between the first layer and outer layer. In various embodiments, the therapy wrap comprises two, three, four, or more layers.

In an exemplary embodiment, the second layer is a heat exchange layer including a heat exchanger. The exemplary heat exchanger is fluid bladder 32. The third layer is a compressive layer including gas pressure bladder 34. As shown in FIG. 2A, for example, the heat exchange layer is not entirely defined by the heat exchanger. One will appreciate that the heat exchanger may extend less than the entire length, width, or thickness of the layer. Similarly, the exemplary pressure bladder extends less than the full length of the compressive layer.

The layers of therapy wrap 115 may include, but are not limited to, a bladder, thermal insulation, padding, a structural member, fluidics, and electrical wiring. In various embodiments, one of the layers is a heat exchange layer defined by a fluid bladder for circulating a heat transfer fluid. In various embodiments, one of the layers is a compressive layer for applying compression to the body. The compression layer may include a gas pressure bladder. Other suitable compression devices may be used such as a spring tensioner, an overwrap layer, an elastomer, a belt or tie wrap, a shrink wrap, a hose-style clamp, and the like. The compression device may include conventional electromechanical devices such as a fabric that constricts in response to an electrical charge or a hose clamp and actuator. In various embodiments, the outer layer is formed of an elastic material to apply compression. Thus, any of the layers may be overlapping in function and position.

In exemplary wrap 115, outer layer 55 is a sleeve cover that covers and protects the inner layers. In various embodiments, the sleeve forms the inner layer and outer layer. In various embodiments, the inner material layer is nylon and the outer material layer is loop material (e.g. pile). The sleeve may be a conventional sleeve such as the devices described in U.S. Pat. No. 7,837,638, the entire contents of which are incorporated herein for all purposes by reference. Suitable materials for the sleeve include, but are not limited to, nylon, spun bonded material (e.g. spun bonded polyethylene), hook and loop material, and more. In various embodiments, the therapy wrap does not include a sleeve. Instead, one or more of the layers may be configured for fastening the wrap to the body part. The outer layer may be configured to act as a cover for the inner layers or no cover may be provided. In various embodiments, the sleeve cover is configured to apply compression to the body part.

In various embodiments, one of the layers comprises a moldable material such as a curable foam or resin. The foam may be molded to the body anatomy by applying the wrap snugly to the body and then curing the material. The layer may include a pouch or bladder to be filled with expanding foam. When the wrap is applied to the body, the foam expands around the body part contour to improve conformance.

In various embodiments, one of the layers is a structural layer comprising a rigid or semi-rigid structural member. The structural layer may be a rigid material such as ABS or metal. The structural member may be any conventional shape for imparting greater rigidity and strength to the portion of the therapy wrap in which it is positioned. In various embodiments, the structural layer includes a structural member only in selected areas. In various embodiments, the structural layer is configured to reduce the risk of kinking or buckling. In various embodiments, the structural member has a predefined shape corresponding to a body part to which the wrap is intended to be applied. A knee wrap, for example, may have a structural member with a predefined contour positioned in the knee cap area to improve conformance to the knee during flexure, or to prevent movement of the joint or body part. In various embodiments, the outer layer comprises a rigid structural material for protecting the inner wrap material.

In various embodiments, the outer layer comprises a clamping device. The clamping device may be configured to grasp the inner layers and clamp the therapy wrap to the body part. For example, a fluid bladder can be secured to a wrist with a C-shaped clamp. The wrap may include the clamping device instead of or in addition to the sleeve cover. In various embodiments, the clamping device is configured to provide a compression for to the body part in addition to or in place of a separate compression device.

In various embodiments, one of the layers is an insulation layer configured for modifying thermal and/or electrical conductivity through the layer. The insulation layer may comprise a thermal insulator. For example, a layer positioned outwardly of the fluid bladder may have a low thermal conductivity to prevent heat exchange of the fluid bladder with the environment. In the case of a therapy wrap with electrical components, an electrically insulating layer may be provided to isolate the electrical components from the other layers and remainder of the wrap. The electrical insulating layer may comprise electrical components isolated within an electrically isolating material or structure.

In various embodiments, one of the layers is configured to provide padding to the body part. The layer may include a foam, a cushion filled with gel or a fluid, an elastomer, and the like. The layer may be configured for protecting the body, for example, by including a shock-absorbing material.

Other layer types and configurations will be understood from the description herein. One will appreciate that the therapy wrap may include additions to or modifications of the layered assembly.

FIG. 2B illustrates a therapy wrap 215 similar to therapy wrap 115. Therapy wrap 215 includes a first layer 250, a second layer 252, and an outer layer 255. Unlike therapy wrap 115, therapy wrap 215 includes a thin film or coating 60 along an inner surface of layer 250.

In various embodiments, the coating is a spray on adhesive or other attachment device. The coating may be a tacky substance applied to the surface to promote contact with the body part and keep the wrap from slipping. An example of a suitable tacky coating is a thin film of a thermoplastic elastomer or thermally-conductive gel. In various embodiments, the coating is applied by dip coating one or more of the other layers. The coating may be configured for protecting the wrap from abrasion, contamination, and other physical contact. Suitable coating materials include, but are not limited to elastomers, resins, wax, sheet metals, and more. The coating may be formed of a spray-on, polyurethane-based material. In various embodiments, coating 60 is configured to be peeled off of a surface of inner layer 250. One will appreciate from the description herein other suitable coatings in accordance with the invention.

FIG. 2C illustrates a therapy wrap 315 similar to therapy wraps 215 and 115 in various respects except therapy wrap 315 includes a cut-out 62 in an inner layer 350. The cut-out is configured to accommodate a portion of a body part 308 to which the wrap is applied. The exemplary cut-out is positioned, shaped and dimensioned to accommodate a feature of the body part. The exemplary therapy wrap is a knee wrap, and the cut-out is configured for accommodating the knee when it is straight, flexed, or both.

Exemplary cut-out 62 is positioned on wrap 315 such that it aligns with the knee cap when applied over the knee. The cut-out may have a shape and dimensions corresponding to the knee cap. The exemplary cut-out is elliptical to correspond to the shape of the knee during flexure. The cut-out also is slightly larger than the size of a typical knee to accommodate larger sizes and reduce the risk of pinching sides of the knee.

Exemplary therapy wrap 315 includes an intermediate layer 352 modified to accommodate a flexed knee. The intermediate layer has a thickness T1 along the middle of the expected knee cap location and a thickness T2 along its remainder. With the exemplary intermediate layer the thickness change is gradual. Exemplary intermediate layer 352 is a fluid bladder. The illustrated shape is formed using conventional techniques such as spot welds to control the maximum inflated thickness or a rigid preformed member to control the shape of the bladder. Examples of spot welding techniques are described in U.S. Pat. No. 7,198,093 and U.S. Patent Pub. No. 2005/0256556 A1 to Schirrmacher et al., the entire contents of which are incorporated herein for all purposes.

One will appreciate, however, that other configurations may be applied. For example, the fluid bladder surface facing the body part may include a coating such coating 60 described above. The wrap may also be provided with an additional layer of padding between the fluid bladder and the body part. Since the body part discussed above is a knee, it may be desirable to provide a thermal insulating layer between the fluid bladder and the body part. Similarly, a cut-out for allowing more direct contact with the fluid bladder may be desirable to increase heat exchange, such as if the body part corresponding to the cut-out includes substantial soft tissue. One or more cut-outs 62 may also be formed in any of the other wrap layers in addition to or besides the inner layer. One will appreciate other features and variations of the therapy wrap for accommodating the shape of a body part based on the description herein.

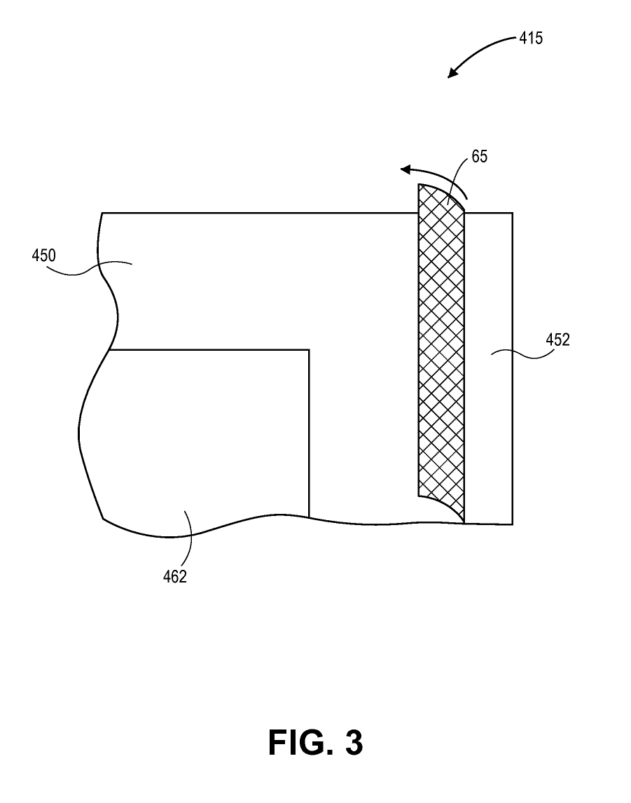

FIG. 3 illustrates a therapy wrap 415 similar to therapy wrap 315 in various respects. Therapy wrap 415 includes a first layer 450 on an inner surface of the wrap. The first layer is shown with an edge peeled back to illustrate a backing 65 and expose a second layer 452 underneath. The backing may be configured similar to coating 60 or any of the layers described above. First layer 450 includes a cut-out 462 which is similar in many respects to cut-out 62 except that cut-out 462 has a polygonal shape.

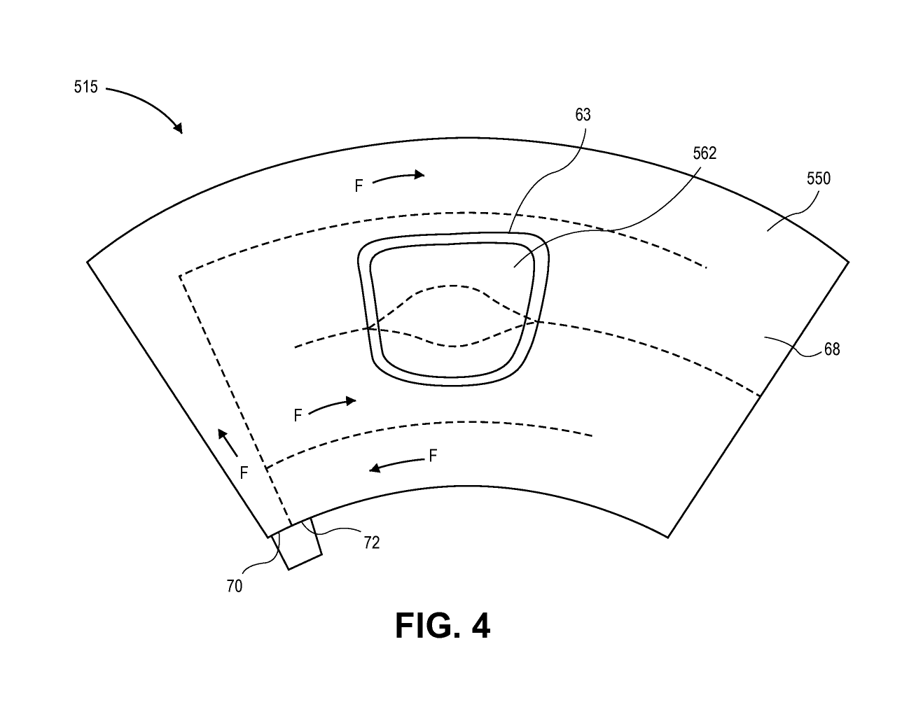

FIG. 4 illustrates a therapy wrap 515 similar to therapy wraps 415 and 315 in various respects. Therapy wrap 515 is a multi-layer wrap assembly shaped for application to a knee or elbow. The therapy wrap includes a cut-out 562 in a first inner layer 550. The exemplary wrap includes padding 63 along a peripheral edge of cut-out 562 to improve comfort for the patient.

FIG. 4 also illustrates an exemplary fluid flowpath 68 of an internal fluid bladder. Fluid is introduced to the flowpath through an inlet 70 and exits through an outlet 72. The exemplary fluid flowpath is essentially circuitous and winds through most of the therapy wrap to make efficient use of the wrap size. The fluid bladder and fluid flowpath is similar in many respects to the subject matter described in U.S. Pat. No. 7,198,093; U.S. Patent Pub. No. 2005/0256556 A1 to Schirrmacher et al., and U.S. application Ser. No. 12/982,266, the entire contents of which are incorporated herein for all purposes. In contrast to conventional fluid bladders, the exemplary fluid flowpath 68 is configured and shaped to accommodate a body part feature thereby improving conformance. More specifically, the exemplary fluid flowpath is directed around the location corresponding to the apex of the bent joint. This may also reduce the risk of obstruction of the flowpath during use. Moreover, in the exemplary case of a knee or elbow, the illustrated design is generally more efficient because the fluid flowpath is generally directed to portions where heat exchange is desirable—the soft tissue—and is directed away from undesirable areas—the area of the bone.

With reference back to FIG. 1, an exemplary method of operating system 5 will now be described. The method will be described in connection with a system for administering cooling therapy and compression; however, the following description is not intended to be exhaustive or to limit the invention to the precise forms disclosed. To the contrary, one will appreciate that many modifications and variations are possible.

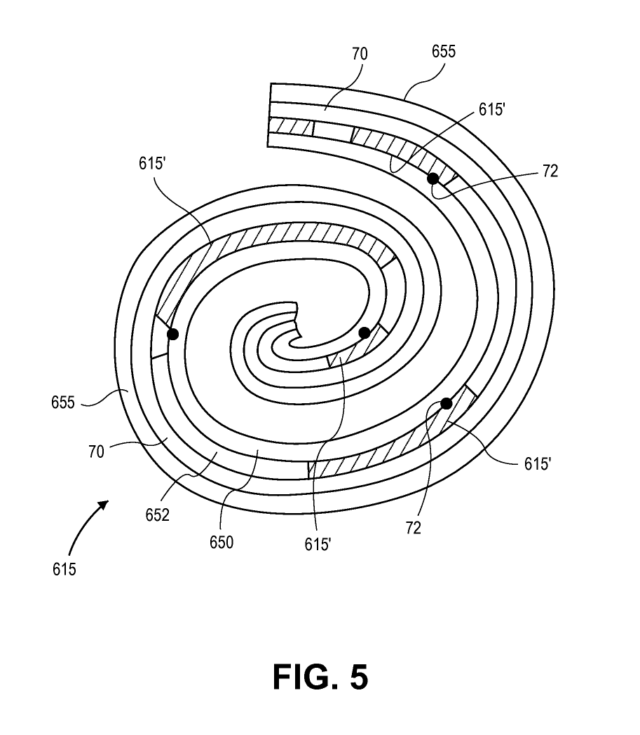

The method will be described in some respects with reference to the exemplary therapy wrap shown in FIG. 5. Therapy wrap 615 is similar to therapy wraps 15, 115, 215, 315, 415, and 515 in various respects. Therapy wrap 615 is a multi-layer wrap including a plurality of therapy wrap devices, generally referred to as heat exchangers and designated 615′, positioned between an optional first inner layer 650 and an inner surface of a substrate 70. In the exemplary embodiment, heat exchangers 615′ include a fluid bladder and gas pressure bladder covered by a sleeve. Each of the bladder assemblies includes a fluid connector 72 for connecting the respective bladders to control unit 10.

On an opposite side of substrate 70 is an outer layer 655. The outer layer may be a cover material. The exemplary outer layer comprises a gas pressure bladder having an outer surface with a covering material such as a fabric sheet.

Therapy wrap 615 is shown in a transport configuration in FIG. 5. The therapy wrap is rolled into a coil. The wrap may be held in the coiled configuration with conventional closures devices. Therapy wrap 615 may be fastened to backer board 33 similar to therapy wrap 15e and unfurled for use. Alternatively, the wrap may be provided as a separate device and laid over the backer board when ready for use.

Unlike many of the therapy wraps described thus far, exemplary therapy wrap 615 includes a plurality of heat exchangers 615′ configured to automatically align with different body parts. In part, substrate 70 allows for positioning the heat exchange devices in predetermined positions. In other words, the heat exchangers are positioned with respect to the body even before they are applied. The heat exchangers are fixed relative to the substrate. In turn, the substrate is configured for a set orientation with the respect to the body. The exemplary substrate has a periphery or outline 37 shaped like a body to make it easier for a user to align a patient on the substrate. As shown in FIG. 1, the substrate and therapy wrap are unrolled onto the backer board. Once the body is laid on the substrate, the target body parts are automatically aligned with the corresponding heat exchange devices. In this manner a therapy wrap with multiple heat exchangers can be quickly and easily applied to a body. This can be particularly helpful when the patient is not compliant or unconscious.

In various embodiments, first inner layer 650 is configured to be removed before use. The exemplary first inner layer is a protective layer to avoid the straps 616 catching on objects during transportation. The exemplary inner layer thus functions like a security seal. To prepare the therapy wrap for use the inner layer is peeled away to expose the heat exchange devices.

Once the patient is positioned on the substrate, heat exchangers 615′ are secured to the predetermined locations on body 8 using straps 16. The system is then operated in a conventional manner using control unit 10. The control unit circulates heat transfer fluid from coolant source 19 to the fluid bladder and inflates the gas pressure bladder with air from gas source 17. Further details regarding operation of the system are described in the applications and patents incorporated herein.

Since exemplary system 5 includes a known set of therapy wrap devices with known specifications, one of skill will appreciate that the system may include a preprogrammed treatment routine tailored to the wraps. This information can be used in combination with the patient information. For example, a user may enter the treatment needed or condition presented, for example, cardiac arrest, and the system automatically selects and runs a predetermined treatment routine. The system may also be programmed to factor in the patient's physiological parameters, for example, height, weight, gender, and fitness level. The system may also monitor the patient's vitals during operation and make necessary adjustments. In another example, a user selects therapy wrap(s) from a set of off-the-shelf wraps for use. The system may be configured to recognize which wrap types are selected and run a predetermined treatment routine. The system may be configured to recognize a wrap using a “handshake” procedure, for example, if a user desires to exchange, add, or eliminate any of the therapy wraps.

The exemplary system is preprogrammed to administer treatment automatically under one of a defined set of conditions. The exemplary system is configured for emergency medical response, for example, by including backer board 33 to carry a patient. The control unit may therefore include a set of therapy conditions for emergency medical thermal therapy. For example, the control unit may be configured to administer therapy under conditions to cool the entire body to reduce the risk of ischemia of limbs, the brain, etc. In various embodiments, the control unit is preprogrammed to operate the therapy device under sufficient conditions to cool the body to a temperature below 96° F., preferably between about 90° F. and about 95° F. The system and control unit may be preprogrammed to administer a variety of other therapy procedures.

In various embodiments, the thermal therapy device is operated under sufficient conditions to lower the body temperature at a first rate for a first period of time and then a faster second rate thereafter. In various embodiments, the thermal therapy device is operated under sufficient conditions to lower the body temperature at a first rate for a first period of time and then a slower second rate thereafter.

In various embodiments, the thermal therapy device is cycled through different treatment conditions. For example, the device may be treated at one temperature for a first period of time and then treated at a lower temperature for a second period of time. In various embodiments, the body is cooled gradually, maintained at a predetermined temperature, and then restored to normal temperature gradually.

In various embodiments, the system adjusts the therapy conditions during use based on information related to the system performance such as elapsed time, flow rate, temperature drop of the cooling source, and more.

In various embodiments, the therapy wraps are operated essentially independently. Each of the therapy wraps may be configured to administer a different treatment under different conditions. The device may apply different levels of cooling in different regions or to different body parts. For example, the device may apply greater or less cooling to the chest area than the wrist area to lower the body's thermoregulation defenses. The device may gradually even out the difference in temperatures as the body approaches the desired internal body temperature.

The exemplary control unit operates until an endpoint is reached. In the case of body cooling therapy, the endpoint may include, but is not limited to, a signal that the desired internal body temperature has been reached, passage of a predetermined amount of time, reaching a desired level of heat exchange, or a stop signal prompted by the user (e.g. user turns off control unit or disconnects a wrap). In general, when the endpoint of the thermal therapy has been reached, the control unit stops the flow of gas and/or application of backpressure to the gas pressure bladder and circulation of fluid to the fluid bladder.

The system may also be controlled manually. System 5 may be configured to operate based on and off modes. Alternatively, a user can control the system using control panel 20 on control unit 10. The control unit may be used to monitor and/or regulate fluid flow through therapy wraps 15.

The control panel may also be configured to allow a user to input information such as information related to the desired therapy, the patient, and the system components being used. The desired therapy information may include, but is not limited to, the type of therapy (e.g. hot, cold, and/or compression), desired treatment time, desired average temperature in the therapy wrap, adjustments to the temperature, desired compression, adjustments to the compression, and patient characteristics such as fitness level, height, weight, and sensitivity. The user may include those other than the person or animal receiving the treatment. For example, a caregiver may select a customized therapy setting or input a patient indication such as ligament tear or cardiac arrest.

Although the method of the invention is described in terms of thermal and/or compression therapy in an emergency medical setting, one will appreciate that the methods and systems of the invention may be configured for administering a variety of treatments to a patient in a variety of settings. Suitable treatment settings include, but are not limited to, a clinic such as a rehabilitation or physical therapy clinic, an operating room (OR), a post-operative setting, a hospital, emergency medical care, and more. Suitable treatments include cooling therapy, heat therapy, compression therapy, and combinations of the same. The system and therapy wrap may also be configured for use in conjunction with other therapies such as electric stimulation therapy, light therapy, introduction of intravenous fluids, and more. Exemplary therapy wraps for combining thermal therapy devices and other therapeutic devices are described in co-pending U.S. App. No. 61/472,596 (attorney matter no. 11185-721.100) filed Apr. 6, 2011, the entire contents of which are incorporated herein for all purposes. The control unit may also be used to control administration of other treatments and functions commonly used in the above treatment settings. Exemplary control units and techniques for controlling the system are described in co-pending U.S. App. No. 61/472,598 (attorney matter no. 11185-722.100) filed Apr. 6, 2011, the entire contents of which are incorporated herein for all purposes.

In various embodiments, the system includes one or more sensors 74 to monitor the system performance. The sensors may acquire data and communicate with control unit 10 using otherwise conventional techniques. Sensors may also be used to monitor the pump, valving system, power source, pneumatic source, and coolant source. In an exemplary embodiment, temperature sensors are positioned in the fluidics of the thermal therapy wrap and/or control unit. A pressure sensor may be used to monitor pressure in the gas pressure bladder. In another example, the control unit may receive a temperature reading at an outlet of the fluid bladder and determine that the inlet temperature needs to be decreased.

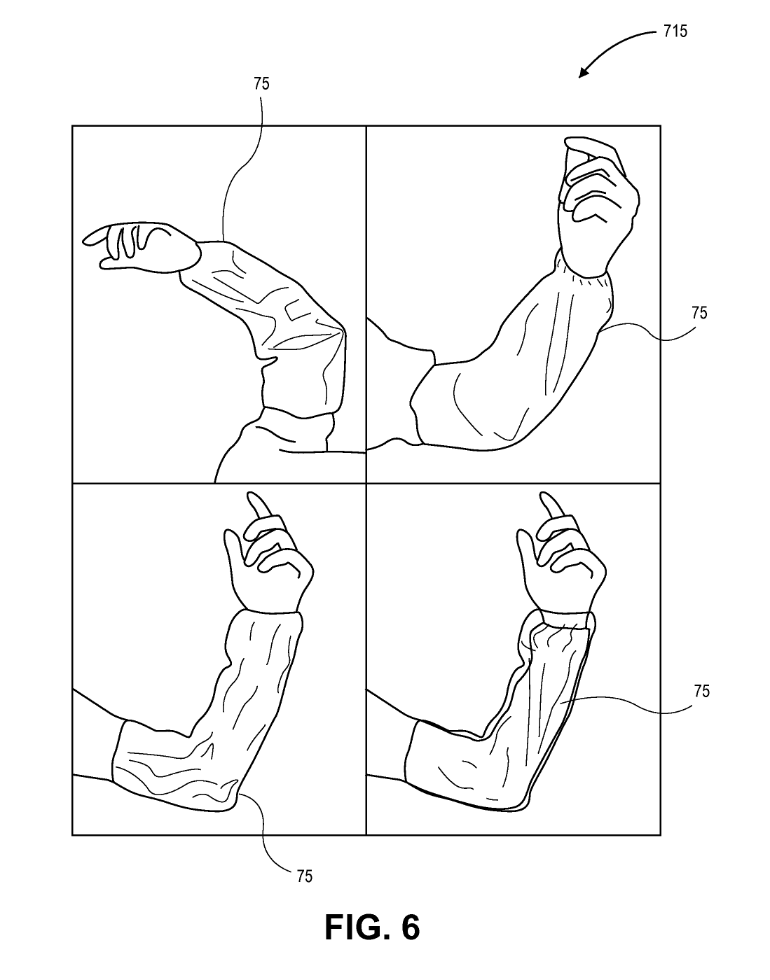

FIG. 6 illustrates a therapy wrap 715 similar to therapy wrap 15, 115, 215, and 315 in various respects except therapy wrap 715 includes a flexible and loose cover 75 in place of an outer sleeve cover. Cover 75 is configured similar to a baggie. The cover may be formed of a plastic such as polyethylene or polyurethane.

Cover 75 is configured to be loose fitting over the internal components and body part. The therapy wrap is attached to the body by first pulling the cover 75 over the body part to be covered. Next, the therapy wrap is attached to the body part in as described above. The sleeve including the fluid bladder and optional compression bladder is fastened around the body part. In one embodiment in which the therapy wrap does not include a sleeve cover, the heat exchange components are attached to the body part and cover 75 using fasteners as described herein.

The exemplary therapy wrap is used in a conventional manner except the cover 75 is disposable. After operation, the user disposes of the cover. The cover ensures that the rest of the therapy wrap remains clean and ready for use. This makes cleaning of the therapy wrap easier and may be useful where hygiene and sterilization are of greater importance.

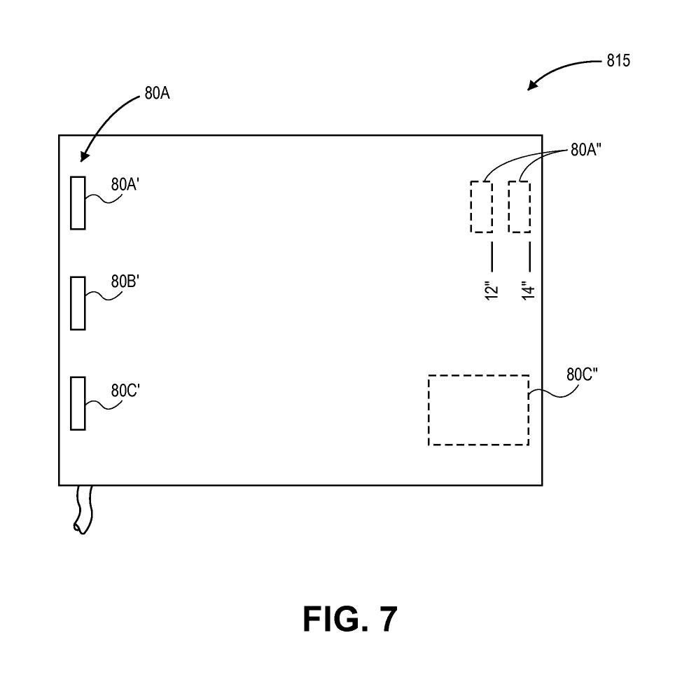

FIG. 7 illustrates another exemplary therapy wrap 815. Therapy wrap 815 is similar to the therapy wrap described above in various respects except therapy wrap 815 includes modified closure mechanisms.

Therapy wrap 815 includes a magnetic closure mechanism 80a having a first portion 80a′ and a second portion 80a″. The exemplary magnetic closure includes a conventional magnet assembly such as a ferromagnetic material or electromagnet. The second portion includes a plurality of attachment points at set increments. The attachment points may be spaced based on the body part to which the wrap is to be applied. For example, a first point may correspond to a larger body part (e.g. a thigh) and a second point may correspond to smaller part (e.g. a bicep). The attachment points may be at increments to allow the attachment to body parts of different people, for example, a smaller person's bicep versus a larger bicep. The wrap may include markers to indicate to the user the diameter of the wrap when it is applied.

Another closure mechanism 80c is similar to closure mechanism 80a except that a second portion 80c″ is configured for infinite adjustment of the wrap size.

Another closure mechanism 80b is configured for mechanical fastening. The closure mechanism may include a hook-and-loop fastener, adhesive, and the like.

One of skill in the art will appreciate that a number of other features and modifications are within the scope of the invention. Variations and modifications of any of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto. Any feature described in any one embodiment described herein can be combined with any other feature of any of the other embodiment whether preferred or not.

For convenience in explanation and accurate definition in the appended claims, the terms “up” or “upper”, “down” or “lower”, “inside” and “outside” are used to describe features of the present invention with reference to the positions of such features as displayed in the figures.

In many respects the modifications of the various figures resemble those of preceding modifications and the same reference numerals followed by apostrophes or subscripts “a”, “b”, “c”, and “d” designate corresponding parts.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

What is claimed is:

1. A system for providing thermal treatment to a body requiring treatment, the system comprising:
at least one therapy wrap comprising a heat exchange layer configured to exchange heat with a body; and
a rigid substrate for supporting the therapy wrap.

2. The system of claim 1, wherein the at least one therapy wrap is secured to the rigid substrate.

3. The system of claim 1, wherein the heat exchange layer comprises at least one a fluid bladder configured to circulate a heat transfer fluid.

4. The system of claim 1, wherein the at least one therapy wrap further comprises:
an insulating layer positioned between the heat exchanger layer and the body to receive treatment;
a compressive layer for applying a compressive force to the body, the compressive layer positioned on an opposite side of the heat exchange layer as the insulating layer.

5. The system of claim 1, wherein the rigid substrate is a rigid brace.

6. The system of claim 5, wherein the substrate is shaped and dimensioned to support a human body.

7. The system of claim 6, further comprising a plurality of therapy wraps.

8. The system of claim 7, wherein the plurality of therapy wraps are each positioned at different locations on the substrate corresponding to different parts of the body.

9. The system of claim 8, wherein each of the plurality of therapy wraps is configured to exchange heat with the animate body at different rates.

10. The system of claim 8, wherein each of the plurality of therapy wraps comprises a heat exchange layer including at least one a fluid bladder configured to circulate a heat transfer fluid, the system further comprising a control unit configured to independently regulate the flow of heat transfer fluid in each of the plurality of therapy wraps.

11. A therapy wrap for providing thermal treatment to a body requiring treatment, the therapy wrap comprising:
a first layer for contacting the body at a treatment site; and
a heat exchange layer positioned outwardly of the first layer, the heat exchange layer including at least one heat transfer device configured to circulate a heat transfer fluid to exchange heat with the body;
wherein the first layer includes a cut-out in a location corresponding to a feature of the body.

12. The therapy wrap of claim 11, wherein the first layer comprises padding.

13. The therapy wrap of claim 11, further comprising a compressive layer including an expandable portion for applying a compressive force to the body, the heat transfer device, or both, the compressive layer positioned outwardly of the heat exchange layer.

14. The therapy wrap of claim 13, wherein the expandable portion includes a gas bladder.

15. The therapy wrap of claim 13, wherein the expandable portion includes expandable foam.

16. The therapy wrap of claim 13, further comprising a structural layer including a rigid member for increasing local bending strength of the therapy wrap.

17. The therapy wrap of claim 16, the rigid member extending along a region of the cut-out, wherein the rigid member has a predetermined shape corresponding to the feature.

18. The therapy wrap of claim 11, wherein the feature is one of a joint and a three-dimensional contour.

19. A system for providing thermal treatment to a body requiring treatment, the system comprising:
a plurality of therapy wraps each configured to attached to a portion of a body and administer thermal therapy at different temperatures.

20. The system of claim 19, further comprising a control unit for controlling administration of thermal therapy using the plurality of therapy wraps.

21. The system of claim 19, wherein the plurality of therapy wraps are configured to attach to at least one of a wrist, a forearm, a foot, a leg, a torso, a thoracic region, a cranial region, a throat region, a limb, and a combination of the same.

22. The system of claim 19, wherein one of the plurality of therapy wraps is configured to administer therapy at a temperature significantly below a normal internal temperature of the body and another of the therapy wraps is configured to administer therapy at a temperature near or about the same as the normal internal temperature.

23. The system of claim 19, wherein one of the plurality of therapy wraps is configured to administer therapy at a temperature significantly below a normal internal temperature of the body and another of the therapy wraps is configured to administer therapy at a temperature warmer than the normal internal temperature.
